(19) United States
(12) Patent Application Publication
(10) Pub. No.: US 2022/0331603 A1
(43) Pub. Date: Oct. 20, 2022

(54) DEVICE FOR EMITTING A MAGNETIC FIELD

(71) Applicant: Biopass S.A., Luxembourg (LU)

(72) Inventor: Daniel Greff, Mere (FR)

(73) Assignee: Biopass S.A., Luxembourg (LU)

(21) Appl. No.: 17/633,109

(22) PCT Filed: Aug. 4, 2020
(86) PCT No.: PCT/EP2020/071926

§ 371 Date: Feb. 4, 2022

(30) Foreign Application Priority Data
Aug. 5, 2019 (FR) FR1908980

(51) Int. Cl. A61N 2/02

(52) U.S. Cl. CPC A61N 2/02

(57) ABSTRACT

The invention disclosure relates to a device for emitting a magnetic field including an insulating support intended to be applied on a part of a person's body, at least two hexagonal antennas, and a power supply source. The device has at least a first antenna and at least a second antenna, that are reversely wound.

### TECHNICAL FIELD

The present invention relates to the general field of the use of electromagnetic waves to act on the metabolism of the human body in order to induce physiological changes, in particular to promote sleep, simulate physical activity or treat adipose overloads in a person.

### BACKGROUND OF THE INVENTION

Reducing fatty mass from the upper layers of the skin by applying a magnetic field has already been proposed. Reference can in particular be made to the documents FR 2,855,415, FR 2,906,727, EP 2,068,810, which describe devices consisting of insulating straps applied to the part of the human body to be treated, these straps being equipped with a conductor folded in successive strands and linked to an alternating voltage generator to create electromagnetic waves.

Devices allowing the stimulation of lipolysis of the fatty visceral and intramuscular mass by emitting low frequency electromagnetic fields have been proposed recently in application EP 2,665,516. These devices comprise rectangular shaped dipolar antennas.

Finally, devices comprising hexagonal coils have been described in applications DE 2010 012 057 and KR 2008 061 463.

However, there is still a need to improve existing devices.

### OBJECT AND SUMMARY OF THE INVENTION

This object is attained thanks to a device for exposing at least a part of a person's body to a magnetic field, said device comprising:

an insulating support intended to be put into contact with a part of the person's body,

at least one antenna set comprising at least a flat dipolar hexagonal antenna, said antenna emitting a magnetic field according to a vector that is perpendicular to a surface of the antenna,

a power supply source that supplies an alternating or pulsed current to the antenna,

the antenna and the power supply source being embedded in the insulating support.

The device of the invention preferably comprises at least one set of hexagonal antennas, said set of antennas comprising at least one hexagonal antenna, on condition that the device comprises at least two antennas coiled in opposite directions. In an antenna set comprising two or more antennas, the antennas are connected in series. In a device comprising at least two sets of antennas, the sets of antennas are connected in parallel.

In a particular embodiment, the invention relates to a device for emitting a magnetic field, comprising:

an insulating support intended to be brought into contact with a part of a person's body,

at least one first antenna and at least one second antenna, each antenna being a coil of conductive metal wire that is wound along the outline of a hexagon, and

a current supply source for the antenna,

the first antenna, the second antenna and the power source being integral with the insulating support,

the device being characterized in that the current is alternating and in that the first antenna and the second antenna are wound in opposite directions.

When the current is alternating, the first antenna therefore emits a magnetic field in opposite direction to the one that is emitted by the second antenna, and the direction of the magnetic field emitted by each antenna alternates according to the frequency of the supply current. This device has several advantages. The fact of combining two coils whose winding direction of the wire is reversed, and of supplying the two coils with an alternating current makes it possible to emit a global magnetic field which crosses the body of a person on which the device is placed, during each half period of the alternating current, and not during a whole period.

### BRIEF DESCRIPTION OF THE DRAWINGS

Other features and advantages of the present invention will become apparent from the description given below, with reference to the accompanying drawings,

FIG. 1 is a view of a person fitted with three devices according to the invention: one device around each arm 200, one device around the waist 100 and one device around each leg 300 comprising several sets of antenna;

FIG. 2 is a detailed view of the device according to the invention that can be applied around the abdominal belt of a person;

FIG. 3 is a view of the device according to the invention comprising two antennas that are coiled in opposite directions, and a diagram representing the profile of the magnetic fields emitted by each of the antennas

FIG. 4 is a view of the part of device 300 in FIG. 1 that is applied around the left leg;

FIG. 5 is a device intended to be applied on the contour of the face,

FIG. 6 is a device in the form of a mat;

FIG. 7 represents an antenna in the form of a regular hexagon having a side A.

FIG. 8 is a device that can be applied around hands.

### DETAILED DESCRIPTION OF THE EMBODIMENTS

The device of the invention comprises at least two hexagonal antennas.

“Antenna of hexagonal shape” is understood to mean a hexagonal flat dipole antenna which emits a magnetic field along a vector perpendicular to its surface. The hexagonal shaped antenna is a coil of conductive metal wire that is wound along the outline of a hexagon. The angles of the hexagon are preferably slightly rounded so as to prevent breaking the conductive wire. The conductive metal line is advantageously drawn along the perimeter of the hexagon and is wound several times around inwards or outwards, without touching, so that the spires are not in contact with each other. In order to limit the density of the current in the device, the electric conductor can comprise a maximum of around ten turns in order to ensure both i) the safety of the user (limit an excessive temperature rise that may lead to burns) and ii) an adequate magnetic field intensity. Each antenna enables to generate a field whereof the form is similar to a cone of hexagonal section. The antennas are preferably is spools in copper wire. Thickness of the metal wire is preferably between 200 and 300 microns.

“Hexagonal” means a geometric shape comprising six sides. The lengths of the sides of the hexagon are preferably equal to within plus or minus 10%. The hexagon is preferably a regular hexagon, with all six sides having the same length. A particular hexagon has a minor axis connecting two opposite sides and a major axis connecting two opposite vertices, the minor axis and the major axis being perpendicular.

In a particular embodiment, the invention relates to a device for emitting a magnetic field, comprising:

an insulating support intended to be brought into contact with a part of a person's body,

at least one first antenna and at least one second antenna, each antenna being a coil of conductive metal wire wound along the outline of a hexagon, and

a current supply source for the antenna,

the first antenna, the second antenna and the power source being integral with the insulating support,

the device being characterized in that the current is alternating and in that the first antenna and the second antenna are wound in opposite directions.

The device of the invention preferably comprises at least one set of antennas, said set of antennas comprising at least one hexagonal antenna, on the condition that the device comprises at least the first antenna and the second antenna.

The device of the invention may comprise at least two sets of antennas, the sets being electrically connected in parallel or in series. It is preferred that the two sets be connected in parallel.

According to a first variant, a set of antennas preferably comprises at least the first antenna and the second antenna, the first antenna and the second antenna being wound in the opposite direction. The first antenna and the second antenna may or may not be directly electrically connected. When the first antenna and the second antenna are part of the same assembly, they are preferably directly electrically connected.

When the antenna set includes more than two antennas, it is preferred that two antennas which are directly electrically connected have a reverse winding direction, In a set of antennas comprising more than two antennas connected in series, it is therefore preferred to connect the antennas by alternating the direction of their winding.

According to a second variant, the first antenna and the second antenna (which have a reverse winding direction) belong to two distinct sets of antennas.

In a set of antennas, the antennas can be arranged in sub-groups of at least two antennas, the distance between adjacent antennas of a sub-group being less than the distance between two antennas of two different sub-groups.

The insulating support can be formed of at least two sheets of a fine soft insulating material, for example two sheets of a polymer material of a thickness being between 200 microns and 300 microns.

The antennas are advantageously placed inside the insulating support so as not to come in contact with the body. they can be for example placed between the two aforementioned sheets of insulating material.

The thickness of the insulating support is chosen in such a way that the energy dissipation which is generated during the operating of the antenna does not cause a too high temperature rise and does not bother the person. The thickness is for example between 400 microns and 600 microns.

The insulating support can take different forms, depending on the part of the body for which the device is intended, For example, the insulating support can be in the form of a belt intended to be wound around a part of the abdomen. The insulating support can be alternatively cut in the form of a strap so as to espouse the contour of the face at the neck. According to other options, the insulating support is in the form of a mat, whereon the person can lie down, or in the form of a bracelet that can be wound around the arm or ankles.

The antennas of the device according to the invention emit a magnetic field directed locally towards certain areas of the body to be treated, namely, in particular the abdominal truss, the face, the arms, the thighs, the calves, the ankles, and the back.

Applying this magnetic field enables to simulate a sports activity, particularly by its action on the calcium channels and thus, without the patient feeling any muscle contractions. This device not only enables to reduce the subcutaneous, visceral and intramuscular fatty mass, but also combats obesity and metabolic syndrome and significantly increases the chances of medically assisted procreation for sterile persons.

The device, when placed on the body of a healthy person, will have a non- therapeutic effect, although acting on the metabolism. It may have an aesthetic effect by reducing my subcutaneous fat, or simulate a sporting activity.

In a person suffering from a disease, in particular a person suffering from obesity, the device will have a therapeutic effect on the disease to be treated.

When the device is an abdominal belt, the main component of the magnetic field emitted by the antenna is not coaxial to the vertebral column of the person. Furthermore, the intensity of the magnetic field to which are exposed the critical areas of the body (heart, lung, brain) is very low, such that the use of the device according to the invention hardly poses any risks for the person's health.

The power supply of the antenna emits a power supply current that can be alternating or pulsed with a frequency between 10 and 100Hz, preferably between 40 and 60 Hz, It can be equal to 16 Hz, 32 Hz, 48 Hz, 64 Hz, 80 Hz or 96 Hz, preferably of the order of 50 Hz. By alternating current, is meant that the value and direction of the instantaneous current change, By alternating current, it is meant that the value and the direction of the current vary as a function of time, preferably according to a sinusoidal function.

When the power supply current is an alternating current, it preferably has a voltage of the order of 5 to 25 V, for example approximately 10 or 20 V, and an intensity of between 0.3 and 1.5 A, for example of the order of 0.5 A. In fact, it has been found that a power supply current exhibiting such characteristics makes it possible to obtain the best results in terms of emitted magnetic field density as well as of energy density, thus, avoiding putting the patient in a situation of electrical or thermal risk.

Advantageously, the antenna is sized to emit a magnetic field with a maximum intensity of 600 microTesla (600 microT, namely 6 Gauss). It has indeed been found that the application of a magnetic field of intensity greater than 600 microT can have, at high doses, damaging secondary effects on the cells of the brain and bones of the person. Thus, the limitation to such an intensity threshold prevents such risks.

The device of the invention may comprise at least three antennas arranged in a paving,

Independently, the first antenna and the second antenna can be aligned along their largest axis or along their smallest axis.

A device of the invention may comprise at least two sets of antennas connected in parallel, each set comprising at least two antennas connected in series and comprising at least the first antenna and the second antenna. hi this case, the device can be in the form of a belt or pants.

A device of the invention can comprise at least two sets of antennas, that the centers of the antennas of the same set are positioned on the same axis, that the axes of the sets are parallel, and that each set comprises at least the first antenna and the second antenna. In this case, the device can be in the form of a belt or pants.

For example, the device is an abdominal belt comprising a rectangular medium and two antenna sets, the antennas of a same antenna set being supplied in series, and two antenna sets being connected in parallel. In this case, the power source current is preferably an alternating current, in a way that the antennas having the same winding direction emit a magnetic field with a direction opposite to that emitted by the antennas wound in the other direction. According to one embodiment, the antennas of a same antenna set are aligned and the two rows of antenna are place in staggered rows.

The device may comprise a set of antennas connected in series comprising at least the first antenna and the second antenna, the antennas of the set of antennas being distributed in groups of three, and the antennas of a group being arranged in a paving.

According to another embodiment, the device is a face strap intended to be worn around the oval of the face, on the neck. The strap comprises a curved medium and has three to five series-connected antenna sets, each antenna set comprising from one to five antennas, preferably three, arranged in a paving pattern. In this antenna set, a clockwise coil antenna can be directly connected to a counter-clockwise coil antenna.

According to another embodiment, the device is a bracelet intended to be worn around the arm or ankle, said bracelet comprising a rectangular medium, the first antenna and the second antenna. The device can comprise a third antenna, the center of which is aligned with the center of the two other antennas.

According to another embodiment, the device is a strap intended to be worn around the hands, said hand strap comprising a rectangular support and a set of antennae comprising three first antennae and three second antennae arranged along the perimeter of the support.

According to a further embodiment, the device is intended to be placed around the leg. For example, it comprises at least two antenna sets, each antenna set comprising at least two antennas, preferably two reverse-wound antennas, the centres of the antennas of a same antenna set being positioned on a same axis, and the axes of the antenna sets being parallel. A particular device comprises three to five antenna sets intended for hip, thigh, calf and/or ankle. For example, the device can comprise five antenna sets: one antenna set of three antennas at the ankle, one antenna set of four antennas at the calf, two antenna sets at the thigh composed respectively of five antennas and three antennas, and one antenna set at the hip composed of two antennas.

The device may comprise a set of antennas comprising at least five first antennas and at least five second antennas, all fed in series and arranged in a paving pattern.

According to a last embodiment, the device comes in the form of a mat whereon the person can lie. The mat can be placed on the mattress of a bed or on the ground. It can serve as a relaxing mat or a mat for gymnastics. The mat will preferably be placed in such a way that the antennas be located in the immediate vicinity of the back, preferably still from the lower back towards the middle of the back. In a particular embodiment, the mat comprises between 10 and 25 antennas, or between 15 and 25 antennas. The device is for example in the form of a rectangular mat that comprises one single antenna set comprising at least ten antennas arranged in a paving pattern and supplied in series. The antennas placed in a paving pattern can define rows of two or three antennas arranged in staggered rows.

The device of the invention can further comprise a module for controlling the alternating current power supply source of the antenna(s) and at least one magnetic field measurement sensor arranged on the medium and connected to said control module. The presence of one or several magnetic field measurement sensors makes it possible to precisely determine the magnetic induction threshold applied to the person. Depending on these measurements, an adjustment can hence be carried out by means of the control module,

The purpose of the devices of the invention is to emit magnetic fields, the features of which (particularly the direction and intensity) aim to reduce the fatty mass present in the targeted parts of the body. This reduction of the adipose overloads is made possible by virtue of the effect of the magnetic field generated by the device according to the invention which simulates a sports activity.

Whichever part of the body on which it is applied, the device according to the invention comprises i) an insulating support, ii) at least one flat dipolar hexagonal antenna that are reversely wound and that emit a magnetic field according to a vector that is perpendicular to its surface, and iii) a current power supply source of the antenna, the power begin preferably alternating,

Each antenna is advantageously sized to expose the body of a person to a magnetic field with a maximum intensity of approximately 600 microT. Once the device is applied in contact with the person, the magnetic field should preferably be 600 microT maximum on the skin's surface and not exceed 1 microT in the middle of the body. Applying a magnetic field with an intensity of over 600 microT can, in high doses, cause harmful secondary effects on the cells of the brain and bones of the person. The limitation to such an intensity threshold of 600 microT can therefore, prevent such risks.

The sides of the hexagonal antennas preferably have a length between 2 and 20 cm, preferably between 3 and 15 cm, and even more preferably between 4 and 7 cm. One side of length of the order of 5 to 6 cm particularly allows for an opening conditioning the rapid drop in power of the magnetic field according to the depth in the body.

According to a particular embodiment, the hexagon is regular, and the length of its side is between 2 cm and 20 cm.

The antennas of a same antenna set can be arranged according different ways, for example being aligned or being arranged according to a paving pattern. They can be aligned according to their smallest axis (in this case two sides of two contiguous hexagons face each other) or aligned according to their largest axis (in this case the summits of two contiguous hexagons face each other).

The antennas of a same antenna set preferably have the same form and the same dimensions, All the antennas of the device preferably have the same form and same dimensions.

The antennas are preferably arranged in such a way that the smallest distance between one side of a first antenna and one side of a second contiguous antenna ranges between 0.5cm and 1.5cm. Thus, the distance between two contiguous antennas — whether they belong or do not belong to the same antenna set — preferably ranges between 0.5 and 2cm, preferably still of the order of 1cm, This distance makes it possible to resolve the safety issues inherent to the device, in terms of electric if the device were to be damaged, as well as in terms of magnetic field and compliance of magnetic powers in limited sections of the body, in sufficient but limited depths.

In one embodiment, the antennas are electrically linked together in such a way that the antennas of a same antenna set are series-supplied. The antenna set preferably comprises at least two series-connected antennas supplied with an alternating current.

When the device comprises two antenna sets, it is preferred that the power supply be carried out in such a way that, the antennas wound in the same direction and belonging to a first antenna set, emit a magnetic field according to a vector that has an opposite direction to the vector of the magnetic field emitted by the antennas wound in an opposite direction and belonging to the second antenna set. The magnetic field lines coming from the antennas of a same antenna set thus, exhibit maximum intensity on the surface of the skin and a lesser one deeper down in the body. In the case where the device is placed on the abdominal truss, the magnetic field generated by such a device allows for optimum treatment of the muscle areas located near the hips and at the abdominal muscles (the intensity of the field being concentrated in these areas). Deeper down in the body, the intensity of the magnetic field being reduced, the organs of the person and particularly the vertebral column are thereby, spared by the magnetic field emission. Moreover, the antennas of the device emit a magnetic field according to a vector perpendicular to their surface, the magnetic field radiated below and above being extremely low. The vital organs of the person such as the heart and lungs are hence, also spared, when the device is placed nearby.

The power supply source preferably outputs an alternating or pulsed current, of which the frequency ranges between 10 and 100Hz, preferably between 40 and 60Hz. It can be equal to 16 Hz, 32 Hz, 48 Hz, 64 Hz, 80 Hz or 96 Hz, preferably of the order of 50 Hz. Furthermore, when the power supply current is alternating, it advantageously has a voltage of between 5 and 25V, preferably between 5 and 15V. It can also be equal to 20 V in certain embodiments. The intensity of the power supply current of the device is preferably between 0.3 and 1.5 A, preferably still, between 0.5 and 1.5 A, more preferentially equal to 0.5 A.

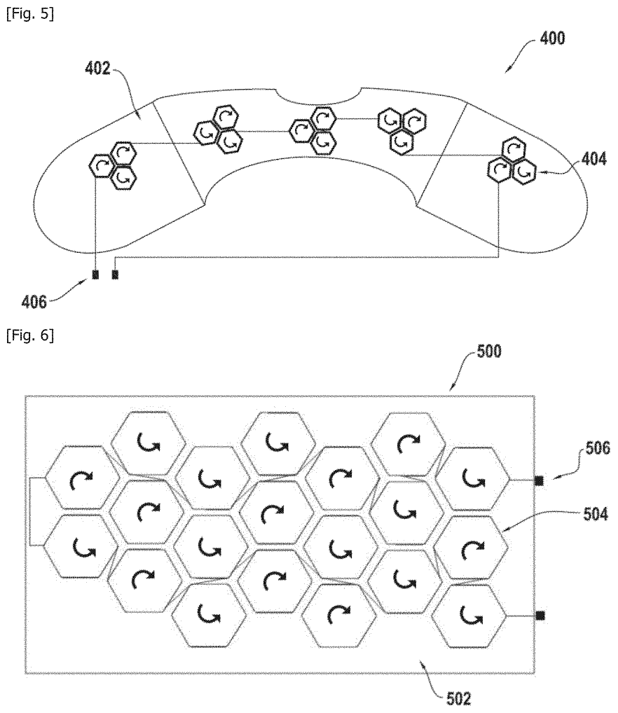

The device according to the invention can be applied hi contact with different parts of a person's body. The figures thus illustrate several devices, namely: a device 100 placed around the abdominal belt, devices 200 placed around the arms, and devices 300 placed around each leg of the person, Device 400 of FIG. 5 is designed to be applied to the neck, to reshape the contour of the face. The device of the invention can also be placed in contact with a person's back, preferably between the lower and the middle of the back, in particular by using the device 500 in the form of a mat of FIG. 6. The device 600 of FIG. 8 is designed to be applied around the hands, and its use has the particular effect of rejuvenating their appearance, in particular by smoothing the skin,

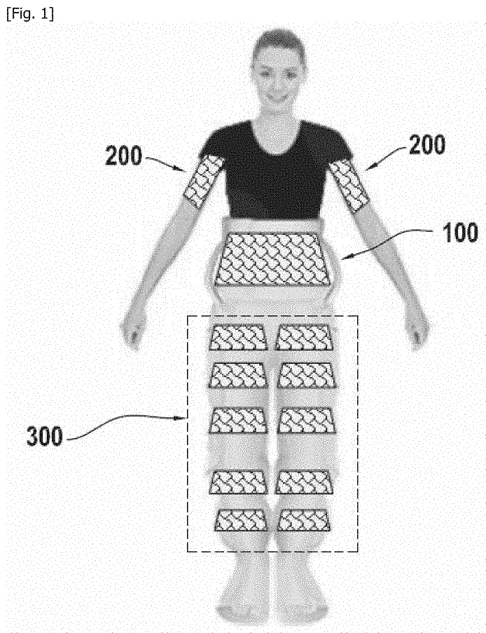

FIG. 1 represents a person on which three devices have been placed: one device 100 in the form of a belt arranged around the abdominal truss, two devices 200 in the form of bracelets arranged around the arms, and a device 300 in the form of a pair of trousers enveloping the legs.

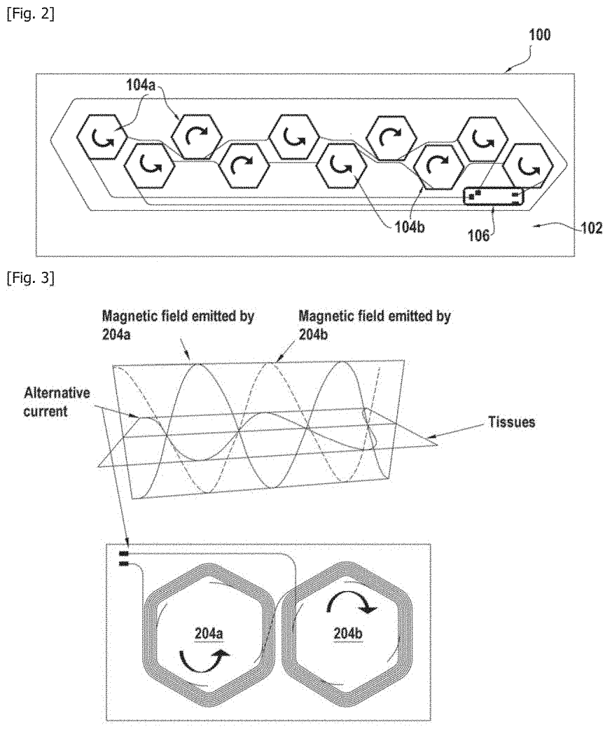

As represented on FIG. 2 the device 100 intended to be applied around the abdominal truss comprises a medium 102 in the form of a belt made from an insulating material, for example PVC, and is able to be closed like a standard belt. The device 100 comprises two antenna sets, the antennas being connected in series and the two antenna sets being connected in parallel. Each set alternates a counter-clockwise wound coil (antenna 104a) and a clockwise wound coil (antenna 104b). The centers of the antennas of one same antenna set are aligned according to the largest axis of the hexagons, and are spaced apart by a pitch selected in such a manner that the two antenna sets can be placed in staggered rows. The smallest distance between an antenna of the first antenna set and an antenna of the second antenna set goes from 0.5 cm to 1.5 cm. The belt can have a more or less large size, for example that of a duvet or cover in which the patient can be placed, Each antenna 104a, 104b is a flat hexagonal antenna having an electromagnetic pole at each of its summits. The antennas 104a, 104b of the abdominal belt device 100 are supplied with an alternating or pulsed current by means of a power supply source 106.

In connection with FIG. 3, now it shall be described the device 200 intended to be arranged around an arm of the person. This device 200 comprises a support 202 made of an insulating material, two flat dipolar hexagonal antennas 204, and an alternating current power supply source 206 of the antenna. The first antenna 204a is a coil whose winding direction is reversed with respect to that of the coil constituting the second antenna 204b. The two antennas 204a and 204b are placed side by side along their smallest axis at a distance of a few centimeters from each other. The arrangement and size of the antennas allows them to emit a first magnetic field with field lines that “pass through” the arm by being cancelled by those of the magnetic field emitted by the second antenna. In this way, the intensity of the magnetic field emitted by the antennas can be adjusted according to the diameter of the arm of the person. If the patient has an arm of small diameter, the influence of the magnetic field originating from the side of the antenna which is opposite to that which is wound, is high and in a direction opposed to the latter. It thus, allows to partially cancel the field emitted towards the tissue of the person. On the contrary, if the arm of the patient has a large diameter, the antenna of the device is arranged nearly vertically with respect to this diameter. The antenna hence comes close to a simple vertical radiant element, of which the distance between the ends becomes insignificant by virtue of the helical form of its winding,

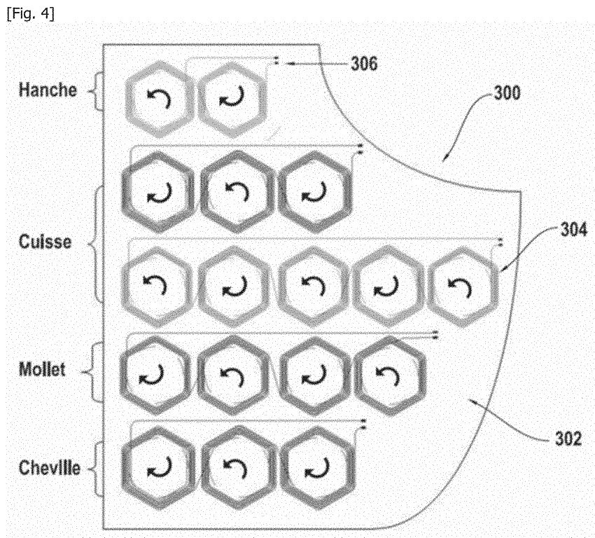

In connection with FIG. 4, shall now be described the left side of the device 300 intended to be arranged around the leg of a person, the device 300 comprising five antenna sets on the left leg and five antenna sets on the right leg. On FIG. 4, five antenna sets 304 are wrapped in an insulating support 302, and are supplied with an alternating current by the power supply source 306. Each set alternates a coiled antenna in one direction and a coiled antenna in the opposite direction. The insulating support of the 300 device is a single piece in the form of pants so that all the antenna assemblies are fixed in relation to each other and can be placed on the patient in a single movement. Alternatively, it is possible to make a separate device for each part of the leg to treat, namely, a device intended to be applied on the hip, one on the top of the thigh, one on the bottom of the thigh, one on the calf, and one on the ankle. A device within the meaning of the invention is therefore preferably in one piece.

FIG. 5 represents a face strap intended to be worn around the oval of the face on the neck. The device comprises a set of flat hexagonal dipole antennas. The assembly comprises five groups of three antennas, the antennas of the same group being arranged in a tiling, and each group comprising two antennas wound in the opposite direction.

The device can also be a mat in compliance with that drawn on FIG. 6 with a scale of 1:6. The mat comprises 20 series-placed antennas of equal dimensions according to a paving pattern defining rows of two or three antennas arranged in staggered rows.

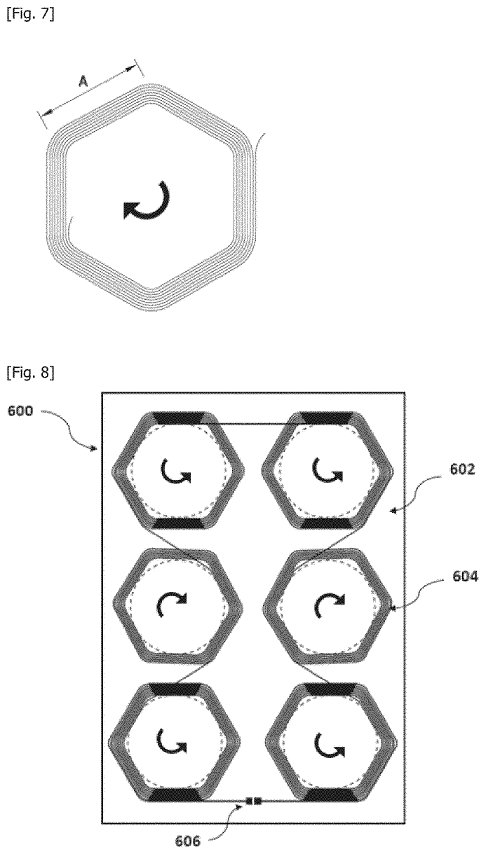

An hexagonal antenna that is part of the device of the invention and the conducting wire of which is wound clockwise, can correspond to the one represented on FIG. 7. Size of the regular hexagon can be 5 cm long.

FIG. 8 shows a device 600, which is intended to be worn around the hand. The device comprises an antenna set 604 comprising six flat hexagonal dipole antennas wrapped in an insulating support 602 and fed in series with alternating current, two directly connected antennas being wound in opposite directions and emitting a magnetic field of opposite direction.

The devices of the invention preferably comprise a module for controlling the alternating power supply current of the antenna (not represented), This control module, thus, makes it possible to control the intensity of the magnetic field emitted for the antenna or antennas of the device.

Furthermore, advantageously still, these devices can comprise at least one sensor for measuring the magnetic field (not represented) arranged on the medium and connected to the control module. The presence of one or several sensors for measuring the magnetic field allows determining with precision the magnetic induction threshold applied to the patient, which corresponds to the optimal intensity of the electric current to choose. Based on these measurements, an adjustment can thereby be carried out by means of the control module.

Particularly, the devices can comprise a means for automatically adjusting the magnetic field intensity emitted for the antenna or antennas according to the users morphology. This is carried out for example by compensating on the magnetic permeability of the persons which the devices can adjust. A dichotomy on the intensity of the magnetic field emitted by the antennas makes it possible to find the ideal operating point. The dichotomy converges when the magnetic field measured from the opposite side of an antenna is equal to, for example 0.01 Gauss (1 microT).

The device of the invention is used for emitting a magnetic field that penetrates the tissue of at least part of at least a member and/or trunk of a person. By “member”, is meant in particular one arm or both arms and one leg or both legs, and particularly a calf or both calves and one thigh or both thighs, and particularly the upper part of the thigh or thighs. By “trunk” is meant in particular the abdominal area, the lumbar region and the neck.

The device of the invention can be applied on at least a part of the body chosen from among the head, the face, the neck, the arms, the back, the waist, the abdomen, the lumbar region, the hips, the thighs, the calves and the ankles. The device can be wound around the body or placed according to a perpendicular axis, parallel or slanted with respect to the axis of the member or the concerned body part.

The low magnetic field level required on the surface of the skin, between a tenth and a few times the earth's magnetic field (0.5 Gauss equivalent to 50 microT), makes it possible to implement these techniques without any risk to the cells of the human body, nor constrains for medical operations.

The device, when placed on the body of a healthy person, will not have any therapeutic effect, although it does act on the metabolism. It may have an aesthetic effect by reducing my subcutaneous fat, have a relaxing effect, or simulate a sports activity.

In a person suffering from a disease, in particular a person suffering from obesity, the device will have a therapeutic effect on the disease to be treated.

The device can therefore be used in methods of aesthetic treatment, methods of therapeutic treatment, methods of relaxation or methods of simulating a sports activity.

The magnetic field applied to living tissue makes it possible, in particular by its action on the calcium channels of the cells, to simulate a sports activity without the patient experiencing any muscle contractions.

Thus, the invention relates to a method for simulating a sports activity on a person. The invention also relates to a method for reducing fatty mass, especially sub- cutaneous, visceral or intramuscular fatty mass, for relaxing or improving the quality of sleep. Each of these three methods includes the implementation of a magnetic field on at least part of at least one member of a person by means of a device such as described previously,

A muscle relaxation method or a method for improving sleep quality, can involve the application of a magnetic field to at least a part of at least one member and/or the trunk of a person by means of the device described previously.

In each of these methods, the device can take the form of a belt, a strap, a bracelet, a cover, a suit, a pair of trousers, a trouser leg, or a mat.

According to a particular embodiment, the method makes it possible to remodel the silhouette or improve the appearance of the body of a healthy person, i.e. who does not have a declared illness in relation to an adipose overload. The device can be used for a non-therapeutic method with the aesthetic aim of reducing the visceral or intramuscular fatty mass, involving the implementation of a magnetic field on at least part of at least one member and/or trunk of a person by means of an aforementioned device, particularly of a healthy person.

The device of the invention allows a reduction in the visceral or intramuscular fatty mass, that is to say at the level of the intramuscular adipocytes. The reduction of the fatty mass in depth allows a very effective improvement of the aesthetics of the person, for example of his silhouette, The devices of the prior art do not make it possible to stimulate lipolysis deep in the tissues. On the contrary, the magnetic field generated by the device of the invention causes the consumption of the fat located in depth, in particular under the dermis, in the viscera or in the muscles. The magnetic field generated by an antenna on the surface of the skin penetrates the body to a given depth, typically of the order of 2 cm to 10 cm, preferably more than 5 cm, over the entire surface of the skin. Its intensity decreases with depth.

Thanks to the device of the invention, it is not necessary to impose on the surface of the skin, a magnetic field of high intensity so as to ensure, deep down, an intensity that is sufficient to activate the biological targets. The devices of the prior art do not make it possible to act at such a depth without putting the person at risk. In fact, the device of the present invention allows for a targeted action of the magnetic field, which substantially radiates between the two planes that are perpendicular to the surface of the antenna and which passes via its lengths. The magnetic field is not diffused on an area of the body that should not be exposed to a magnetic field, such as the heart, the brain or the lungs.

The device can be used for treating or preventing disorders including sterility, obesity and metabolic syndrome, and allows in particular to prevent type2 diabetes, cardiovascular disorders, and cerebrovascular accidents.

A device such as the one described previously can be used in the treatment sterility, obesity and metabolic syndrome.

Metabolic syndrome detected in a person without any particular declared symptoms corresponds to a higher risk of metabolic disorganisation or illness than a person said to be in good health. Thus, metabolic syndrome can be defined as a risk of cerebrovascular accident that is three-times higher than for a person who is in good health. Metabolic syndrome describes a state which is considered as prefiguring several severe illnesses including: type2 diabetes (DT2), cardiovascular disorders, and cerebrovascular accidents (CVA).

The device of the invention can also help prevent or combat obesity, particularly abdominal obesity, and consequently illnesses linked to obesity.

A magnetic field exhibiting the aforementioned intensity and frequency also has a stimulating/improving activity on ovogenesis and spermatogenesis. Thereby, the use of the device of the invention can lead to or increase ovogenesis and/or spermatogenesis. The device therefore makes it possible to treat sterility, to increase fertility, and to prevent a risk of sterility, in particular when it results from obesity or a high risk of obesity against which it is necessary to fight. The magnetic field generated by the device described above can improve fertility in women or men with a Body Mass Index (BMI) greater than or equal to 30.

“Treatment or prevention of sterility” means the stimulation or increase of oogenesis or spermatogenesis, in particular where this is non-existent or where it is insufficient. The treatment of infertility leads to an increase in the pregnancy rate in people following a medically assisted procreation protocol.

Based on the required results, the parameters pertaining to antenna dimension, electric current intensity and voltage for supplying the antenna as well as magnetic field exposure duration sessions and the number of sessions can be adapted.

According to an advantageous embodiment, a control of the dosage of a magnetic field applied on the patient can be performed. This dosage control is particularly performed by regulating the intensity of the magnetic field. This dosage control is easily carried out by varying the intensity of the electric current supplying the antenna and generating the magnetic field, which typically ranges between 0.3 and 1.5 A. The dosage of a magnetic field applied on a person, particularly with the purpose of aesthetic or therapeutic care, can be carried out by placing a magnetic wave sensor in the immediate vicinity of a radiating antenna, said magnetic wave sensor communicating with a magnetic field control module, said control module communicating with one or several radiating antennas, and the measuring carried out by the control module of the magnetic permeability of the person so as to adapt the magnetic field emitted by the radiating antenna(s) according to the individual profile, particularly by decreasing or increasing the current intensity. This also makes it possible for a dosage in intensity and in duration of the magnetic field applied to the person.

A measurement of the response of the tissue of a person in contact with a magnetic field, in particular with the aim of optimising an aesthetic or therapeutic treatment by magnetic field, can include varying frequencies of the electromagnetic field, preferably between 10Hz and 10kHz, facing one or several parts of one or several members and/or one or several parts of the trunk of a person, and the measurement of the electromagnetic field to estimate the response of the tissue of a person. The electric field component, present in any electromagnetic field, becomes significant from 1 KHz, whereas at 50Hz the electromagnetic field is especially characterised by a predominant magnetic field. This electric field thus, makes it possible to carry out a sharp measurement of the magnetic permeability.

This measurement makes it possible in particular to estimate the fatty mass and/or the muscle mass so as to in particular adapt the intensity and duration of the magnetic field applied to the person.

The measurement can be for example implemented by making an alternating current pass through the antenna sets, then successively or simultaneously shutting off one or several antennas to measure the electromagnetic field emitted by the remaining antennas, Thus, by measuring the electromagnetic field emitted through the tissue of a person, for example by positioning the electromagnetic sensor in a diametrically opposed manner to the emitting antenna of the field to be measured, the detected electromagnetic field modelises the magnetic permeability of the person. In the case of the device of FIG. 2, one can easily only let the dorsal antenna emit and measure the field that is received in the abdominal area. A device in the form of a belt can hence comprise an electromagnetic sensor arranged in a manner that is diametrically opposed to at least one antenna when the belt is placed in position on the patient, The electromagnetic sensor can be one of the opposite antennas, but used for receiving in order to detect the signal level passing through the patient.

It is thereby possible to distinguish dielectric permittivity ratios of several orders of magnitude between 10 Hz and 10 KHz, which can actually be measured between 1 KHz and 10 KHz, It is possible to produce a variation curve of dielectric permittivity of the fat (measured on the chest for example) according to the frequency and another variation curve of dielectric permittivity of the muscle tissue according to frequency. The presence of a fat-type curve slope rather than a muscle-type curve slope makes it possible to evaluate the ratios of tissue to be treated. This information is complemented by knowing the weight and size of the person.

The estimation of the response of the tissue of the person can also be carried out by varying the frequencies of the magnetic field on one or several parts of one or several members and/or parts of the trunk of the person. For example, the measurement is performed via a magnetic sensor, and for example a Hall effect or magnetoresistive sensor, notably to determine the maximum and/or optimum power threshold of the magnetic field to be applied to the person.

The device of the invention makes it possible to emit an alternating magnetic field targeting certain areas of the body, and particularly located in areas of high density of muscle and/or viscera. The action of the magnetic field consists in interacting with certain elements involved in the cycles of consumption of energy stored in the body (fat). Particularly, organic cycles linked to calcium are overactivated.

The invention is illustrated by the following Example.

Example 1: Device of the invention and comparison with the prior art 1. Description of the devices

Initially, several devices according to the invention were applied to a first person:

a lap belt as described in FIG. 2,

a bracelet around each arm as described in FIG. 3,

an envelope around each leg as shown in FIG. 4.

on the contour of the person's face, a device as described in FIG. 5.

A second person was laid down on a mat as shown in FIG. 6.

The hexagonal antennas of these five devices were all regular hexagons with sides of 5 cm.

Secondly, for comparison, several devices from application EP 2,665,516 of the prior art were applied to the first person, namely:

an abdominal belt comprising two parallel sets of three flat rectangular dipole antennae arranged along the length of the belt,

a belt around each arm and around each ankle, said belt comprising a set of two parallel rectangular dipole flat antennas arranged in the direction of the length of the belt,

two belts around each leg including a belt on the upper thighs and a belt on the calves, each belt comprising two flat rectangular dipole antennas parallel in the direction of the length,

a strap around the oval of the face, including two flat rectangular dipole antennas,

The flat rectangular dipole antennas were all the same size (35 cm long and 3 cm wide).

The supply current of the devices of the invention and of the devices of the prior art was an alternating current of 50 Hz, 0.5 A and 10 V.

2. Measurement of the intensity of the magnetic field emitted

For each of the devices described previously, the intensity of the magnetic field emitted at three different points was measured with a pre-calibrated GM07 reference Gaussmeter, then the average of the three measurements was calculated,

The results are shown in Table 1 below.

TABLE 1
Magnetic Field (Gauss)

| | Prior Art | | | | Invention | | | |
|---|---|---|---|---|---|---|---|---|
| | Point | Point | Point | | Point | Point | Point | |
| Device | 1 | 2 | 3 | Average | 1 | 2 | 3 | Average |
| Arm Device | 0.6 | 0.5 | 0.7 | 0.6 | 3.5 | 3.39 | 3.21 | 3.36 |
| Abdominal Belt | | | | | | | | |
| Device | | | | | | | | |
| Antenna set n.1 | 1 | 1.1 | 1 | 1.03 | 4.07 | 3.90 | 3.65 | 3.87 |
| Abdominal Belt | | | | | | | | |
| Antenna set n.2 | 0.7 | 0.7 | 0.8 | 0.73 | 3.2 | 3.8 | 3.75 | 3.58 |
| Abdominal Belt | | | | | | | | |
| Leg Device | | | | | | | | |
| Antenna set n.1 | 0.8 | 1 | 0.9 | 0.9 | 5.07 | 4.85 | 5.26 | 5.06 |
| Thigh | | | | | | | | |
| Antenna set n.2 | 0 | 0 | 0 | 0 | 4.4 | 4.64 | 5.43 | 4.82 |
| Thigh | | | | | | | | |
| Antenna set Calf | 0.9 | 0.6 | 0.7 | 0.73 | 3.15 | 4.20 | 4.50 | 3.95 |
| Antenna set Ankle | 0 | 0 | 0 | 0 | 2.96 | 3.25 | 4.11 | 3.44 |
| Device Face | 0.3 | 0.5 | 0.4 | 0.4 | 1.5 | 2.5 | 2 | 2.00 |
| Device Mat | 1 | 1.1 | 1 | 1.03 | 4.5 | 6.0 | 3 | 4.50 |

The value of the magnetic field of the device of the invention is higher than that of the device of the prior art,

In addition, the devices of the invention make it possible to cover 70% of the body, whereas the device of the prior art only makes it possible to cover 30% of the body.

1. A device for emitting a magnetic field comprising:
an insulating support intended to be put into contact with an associated part of a person's body;
at least one first antenna and at least one second antenna, each antenna being a coil of conductive metal wire wound along the outline of a hexagon;
a power source with antenna current;
the at least one first antenna, the at least one second antenna and the power source being integral with the insulating support,
wherein the antenna current is alternating and the at least one first antenna and the at least one second antenna are wound in opposite directions.

2. The device according to claim 1, wherein the hexagon is regular, and that the length of its side is between 2 cm and 20 cm.

3. The device according to claim 1, wherein said device comprises at least three antennas arranged in a paving.

4. The device according to claim 1, wherein the at least one first antenna and the at least one second antenna are aligned along their major axis.

5. The device according to claim 1, wherein the at least one first antenna and the at least one second antenna are aligned along their shortest axis.

6. The device according to claim 1, wherein the at least one first antenna and the at least one second antenna are directly electrically connected.

7. The device according to claim 1, wherein the associated part of the person's body is chosen from the group consisting of head, face, neck, arm, back, waist, abdomen, lumbar region, hip, thigh, calf and ankle.

8. The device according to claim 1. in the form of a belt or trousers, wherein said device comprises at least two sets of antennas connected in parallel, in that each set comprising at least two antennas connected in series and hi that each set comprises at least the at least one first antenna and the at least one second antenna.

9. The device according to claim 1, wherein said device is in the form of a face strap comprising a set of antennas connected in series comprising at least the at least one first antenna and the at least one second antenna, the antennas of the set being distributed in groups of three, and the antennae of a group being arranged in a paving.

10. The device according to claim 1, wherein said device is in the form of a rectangular mat on which the person can lie down, and wherein said device comprises a set of antennas comprising at least five first antennas and at least five second antennas, all fed in series and arranged in a paving.

11. The device according to claim 1, wherein said device is in the form of a hand strap intended to be worn around the hands, said hand strap comprising a rectangular support and a set of antennae comprising three first antennas and three second antennas arranged along the perimeter of the support, the first antenna alternating with the second antenna in the assembly.

12. The device according to claim 1, wherein said device is in the form of a bracelet intended to be worn around the arm or the ankle, said bracelet comprising a rectangular support, the at least one first antenna and the at least one second antenna.
